(19) United States
(12) Patent Application Publication
(10) Pub. No.: US 2024/0081851 A1
(43) Pub. Date: Mar. 14, 2024

(54) ROTARY SHAVER ARRANGEMENT FOR A SURGICAL INSTRUMENT

(71) Applicant: GYRUS MEDICAL LIMITED, Cardiff (GB)

(72) Inventor: James Alan DICKSON, Cardiff (GB)

(73) Assignee: GYRUS MEDICAL LIMITED, Cardiff (GB)

(21) Appl. No.: 18/241,646

(22) Filed: Sep. 1, 2023

Related U.S. Application Data
(60) Provisional application No. 63/404,821, filed on Sep. 8, 2022.

(51) Int. Cl. A61B 17/32; A61B 18/12; A61B 18/14

(52) U.S. Cl. CPC A61B 17/32002; A61B 18/1206; A61B 18/1482; A61B 2017/320064; A61B 2018/00577

(57) ABSTRACT

A rotary shaver arrangement for a surgical instrument includes an outer tubular member with a first cutting window at the distal end thereof, an inner tubular member providing a central suction lumen and rotatably mounted in a central passageway of the outer tubular member, and the inner tubular member having a second cutting window at the distal end of the inner tubular member. The first and second cutting windows are arranged such that: (i) when the inner tubular member is rotated to a first range of angular positions, the first and second cutting windows overlap to form an opening of the central suction lumen; and (ii) when the inner tubular member is rotated to a second range of angular positions, the first and second cutting windows do not overlap. The first and/or second cutting window includes a spline-shaped region to permit the second range of angular positions to be greater than if the spline-shaped region was notch-shaped having angular discontinuities.

### TECHNICAL FIELD

Embodiments of the present disclosure described herein relate to surgical devices, and in particular to a rotary shaver arrangement for a surgical instrument, an end effector for an electrosurgical instrument, an electrosurgical instrument, and an electrosurgical system.

### BACKGROUND TO THE INVENTION

Surgical instruments, including radio frequency (RF) electrosurgical instruments, have become widely used in surgical procedures where access to the surgical site is restricted to a narrow passage, for example, in minimally invasive “keyhole” surgeries. Electrosurgical instruments provide advantages over traditional surgical instruments in that they can be used for coagulation and tissue sealing purposes. Surgical apparatus used to shave, cut, resect, abrade and/or remove tissue, bone and/or other bodily materials are known.

In some electrosurgical instruments, e.g., shaver instruments, the instrument can include a cutting surface, such as a rotating blade disposed on an elongated inner tubular member that is rotated within an elongated outer tubular member having a cutting window. The inner and outer tubular members together form a surgical cutting instrument or unit. In this application, the inner and outer tubular members are also referred to as inner and outer blades. In general, the elongated outer tubular member includes a distal end defining an opening or cutting window disposed at a side of the distal end of the outer tubular member. The cutting window of the outer tubular member exposes the cutting surface of the inner tubular member (located at a side of the distal end of the inner tubular member) to tissue, bone and/or any other bodily materials to be removed. A powered handpiece is used to rotate the inner tubular member with respect to the outer tubular member while an outer tubular member hub (connected to the proximal end of the outer tubular member) is fixed to the handpiece and an inner tubular member hub (connected to the proximal end of the inner tubular member) is loosely held in place by the powered handpiece.

In some instruments the inner tubular member is hollow and has a cutting window on a side surface of its distal end such that tissue, bone, etc. will be cut or shaved as the cutting window of the inner tubular member aligns with and then becomes misaligned with the cutting window of the outer tubular member as the inner tubular member is rotated within the outer tubular member. In this regard, it can be said that the cutting device removes small pieces of the bone, tissue, etc. as the inner tubular member is rotated within the outer tubular member.

In some instruments a vacuum is applied through the inner tubular member such that the bodily material that is to be cut, shaved, etc. is drawn into the windows of the inner and outer tubular members when those windows become aligned, thereby facilitating the cutting, shaving, etc. of the tissue, which then travels through the inner tubular member due to the suction.

Some instruments, e.g., wet-field RF hand instruments for arthroscopy, use a saline suction pathway at the distal tip during either ablating or coagulating tissue. In general terms, suction provides the following benefits:
(i) Cools the RF tip by drawing colder saline over the hot RF tip during use;
(ii) Helps to remove ablated tissue debris from surgical site;
(iii) Removes bubbles to improve joint visibility; and
(iv) Positive effect on the formation of plasma at the tip.

Many times during surgery, the surgeon wishes to apply RF energy to either coagulate bleeding vessels, or ablate tissue in the surgical site without performing cutting with a shaver instrument. This usually is done by withdrawing the shaver instrument and inserting a dedicated RF ablation/coagulation/suction device (for example, a RF wand which is a tube to which suction is applied). However, exchanging the surgical tool for the dedicated RF wand is time-consuming. Furthermore, insertion and removal of instruments into the patient can cause trauma and irritation to the passage of the patient, and thus it is desirable to minimize the number of times that surgical instruments need to be withdrawn and inserted/reinserted into the patient.

An RF shaver has both shaving and RF energy capabilities. In RF shaver electrosurgical instruments, the shaver side (the side on which the tissue, bone, etc. will be cut or shaved as the cutting window of the inner tubular member aligns with and then becomes misaligned with the cutting window of the outer tubular member as the inner tubular member is rotated within the outer tubular member) is opposite an RF side which has an electrode assembly with an active electrode for tissue treatment. The RF side is provided with a suction aperture.

An RF shaver electrosurgical instrument which optimises suction flow is desired.

### SUMMARY OF THE DISCLOSURE

The present disclosure optimises the RF suction flow path for an RF shaver instrument by providing a rotary shaver arrangement for a surgical instrument which prevents an unwanted secondary suction flow path from forming on the shaver side of the instrument at a wide range of angular positions of the inner tubular member. The angular positions of the inner tubular member may also be described as relative angular displacements between the inner and outer tubular members. The present disclosure achieves this by having the blade geometry close off the secondary unwanted suction flow path at a wider range of angular tolerance, without increasing the assembly complexity or component cost significantly. An end effector including the rotary shaver arrangement of the present disclosure is capable of different operations, including mechanical cutting of tissue, and electrosurgical ablation, sealing and/or coagulation of tissue.

Embodiments of the present disclosure provide a rotary shaver arrangement for a surgical instrument, the rotary shaver arrangement having a spline-shaped region in the cutting window of the inner and/or outer tubular member. The smooth nature of the spline-shaped region increases the range of angular positions at which an opening of the central suction lumen is created via the cutting windows of the rotary shaver. This is in comparison to a rotary shaver arrangement having a notch-shaped region in the cutting window of the inner and/or outer tubular member. The increased range of tolerance of mis-antialignment allows the user to close the opening of the central suction lumen with ease as less precision is required for the cutting windows to not overlap. “Anti-alignment” is defined herein as being aligned in an opposite direction, for example the cutting windows of the inner and outer tubular members are perfectly anti-aligned if they are facing in exactly opposite directions to each other i.e. facing 180° away from each other, and thereby facing in diametrically opposed directions away from each other. Therefore, in this application this refers to the first and second cutting windows being antialigned such that the first cutting window faces in a first direction and the second cutting window faces in a second direction precisely opposite the first direction. In contrast, the term “mis-antialignment” is hereby defined as being incorrectly antialigned, i.e., the first and second cutting windows are not exactly facing in opposite directions i.e. are not perfectly diametrically opposed, but are mis-antialigned, for example by having one window being slightly less or slightly more than 180° rotationally displaced from the other window.

In view of the above, from a first aspect, the present disclosure relates to a rotary shaver arrangement for a surgical instrument, the rotary shaver arrangement comprising: an outer tubular member with a first cutting window at the distal end thereof; and an inner tubular member rotatably mounted in a central passageway of the outer tubular member, the inner tubular member providing a central suction lumen, the inner tubular member having a second cutting window at the distal end of the inner tubular member. Wherein, the first and second cutting windows are arranged such that: (i) when the inner tubular member is rotated to a first range of angular positions, the first and second cutting windows overlap to form an opening of the central suction lumen; and (ii) when the inner tubular member is rotated to a second range of angular positions, the first and second cutting windows do not overlap and thus do not form the opening of the central suction lumen, and wherein one or more of the following is true:
(i) the first and/or second cutting window comprises a first shaped region, the first shaped region being a spline-shaped region to permit the second range of angular positions to be greater than if the first shaped region was notch-shaped having angular discontinuities (i.e., the range of positions where the cutting windows do not overlap is a larger range of positions);
(ii) wherein, the first and/or second cutting window comprises a spline-shaped region such that the second range of angular positions is greater than 12° (i.e., the second range spans a range of greater than 12°, e.g., from 174° to 186°); or
(iii) wherein, the first and/or second cutting window comprises a spline-shaped region such that the inner tubular member can be rotated more than 6° from a position where the first and second cutting windows are antialigned without the opening of the central suction lumen forming (i.e., the inner tubular member can be rotated beyond 186° relative to the outer tubular member without the opening forming, wherein a relative angle of 0° refers to the cutting windows being exactly aligned (i.e., facing the same direction)).

Options (i), (ii) and (iii) all describe that the first and/or second cutting window comprises a spline-shaped region such that the opening of the central suction lumen is prevented from forming even if the first and second cutting windows are significantly mis-antialigned. This means that even if the first and second cutting windows (which ideally should be facing in opposite directions, i.e., have a relative angular displacement of 180°) are incorrectly positioned such that the relative angular displacement of the cutting windows is 150°, for example, the opening of the central suction lumen still does not form. In other words, the angular sensitivity to opening an unwanted suction pathway is drastically reduced. This optimises suction flow, especially during RF use of a dual-sided RF shaver device.

The notch-shaped region may comprise at least two vertices. The notch-shaped region may be such that a projection of the distal end of the inner and/or outer tubular member along the longitudinal axis of the inner and/or outer tubular member comprises at least one linear portion.

The position where the first and second cutting windows are antialigned is defined as the position where the inner tubular member is positioned at a relative angular position of 180° to that of the outer tubular member.

In some embodiments, the inner tubular member can be rotated more than 10, 15, 20, 25, 30 or 33 degrees from the position where the first and second cutting windows are antialigned without the opening of the central suction lumen forming.

In some embodiments, the second range of angular positions is greater than 15, 20, 25, 30, 40, 50, 60 or 66 degrees.

In some embodiments, the second range of angular positions includes the first and second cutting windows being antialigned, i.e., the inner tubular member is positioned at a relative angular displacement of 180° to the outer tubular member.

In some embodiments, the first range of angular positions includes the first and second cutting windows being aligned, i.e., the inner tubular member is positioned at a relative angular displacement of 0° to the outer tubular member.

In some embodiments, the second range of angular positions comprises the inner tubular member being positioned at a relative angular position of 170° to 190°, preferably 160° to 200°, more preferably 150° to 210°, more preferably 147° to 213°, to that of the outer tubular member.

In some embodiments, the spline-shaped region is such that a projection of the distal end of the inner and/or outer tubular member along the longitudinal axis of the inner and/or outer tubular member comprises a continuous curve and/or non-linear cut out. In some embodiments, the projection is part-oval.

In some embodiments, the first and/or second cutting window has at least one sharpened edge to form a cutting blade.

In some embodiments, the arrangement is such that, when in use, rotation of the inner tubular member within the outer tubular member causes a tissue cutting action of the cutting blade interacting with the second and/or first cutting window.

In some embodiments, the spline-shaped region is U-shaped. In contrast, the notch-shaped region may be described as an upside down Π shape. The notch-shaped region has harsh corners (or angular discontinuities) rather than the smooth profile of the U.

In some embodiments, when the first and second cutting windows align, they form a substantially oval-shaped interface. In contrast, when the first and second cutting windows of the notch-shaped arrangement align, they form a substantially rectangular-shaped interface.

From a second aspect, the present disclosure relates to an end effector for an electrosurgical instrument, the end effector comprising a rotary shaver arrangement according to any of the above-described embodiments; and a radio frequency (RF) arrangement including an active electrode comprising a suction aperture in fluid communication with the central suction lumen.

In some embodiments, the end effector is arranged such that the RF arrangement is positioned on a first side of the end effector, and the rotary shaver arrangement is positioned such that the direction of tissue shaving of the rotary shaver arrangement is on a second side of the end effector, the second side being opposite the first side. In other words, the end effector is a dual-sided RF shaver.

From a third aspect, the present disclosure relates to an electrosurgical instrument comprising: an end effector according to the any embodiment of the second aspect described above; and an operative shaft having RF electrical connections operably connected to the active electrode, and drive componentry operably connected to the rotary shaver arrangement to drive the rotary shaver arrangement to operate in use.

From a fourth aspect, the present disclosure relates to an electrosurgical system, comprising: an RF electrosurgical generator; a suction pump; and an electrosurgical instrument according to the third aspect, the arrangement being such that in use the RF electrosurgical generator supplies an RF coagulation or ablation signal via the RF electrical connections to the active electrode and the suction pump supplies suction via the central suction lumen connecting the suction aperture located within the electrode to the suction pump.

From a fifth aspect, there is provided a method for processing an instrument for surgery, the method comprising: obtaining the rotary shaver arrangement of the first aspect; sterilizing the rotary shaver arrangement; and storing the rotary shaver arrangement in a sterile container.

### BRIEF DESCRIPTION OF THE DRAWINGS

Embodiments of the invention will now be further described by way of example only and with reference to the accompanying drawings, wherein:

FIG. 1 is a schematic diagram of an electrosurgical system including an electrosurgical instrument;

FIG. 2 is a perspective view of a dual-sided RF shaver device with a secondary unwanted suction flow path (i.e., the cutting windows have slightly overlapped to form an opening of the central suction lumen) during RF use;

FIG. 3 illustrates an inner blade geometry arrangement for multi-purpose blades, e.g. RF shavers;

FIG. 4 shows partial cross-sectional views of the same dual-sided RF shaver device as FIG. 2;

FIG. 5 is a comparison between a perspective view of inner blade geometry illustrating embodiments of the present disclosure (FIG. 5A) and a perspective view of another inner blade geometry arrangement (FIG. 5B);

FIG. 6 is a perspective view of an electrosurgical instrument illustrating embodiments of the present disclosure;

FIG. 7 is a comparison between side views and an end view of an electrosurgical instrument illustrating embodiments of the present disclosure (FIG. 7A) and side views and an end view of an electrosurgical instrument arrangement (FIG. 7B), wherein all the views show the inner blade in the fully open position (i.e., the cutting windows fully overlap to form an opening of the central suction lumen);

FIG. 8 is a comparison between a partial cross-sectional view of inner blade geometry illustrating embodiments of the present disclosure (FIG. 8A) and a partial cross-sectional view of an inner blade geometry arrangement (FIG. 8B), the difference in angle tolerances before the unwanted secondary suction flow path forms can be seen; and

FIG. 9 is a comparison between a projection of the distal end of the inner blade along the longitudinal axis of the inner tubular member in accordance with embodiments of the present disclosure (FIG. 9A) and a projection of the unmodified blade arrangement (FIG. 8B), the difference in shape can be seen.

### DESCRIPTION OF THE EMBODIMENTS

The Electrosurgical System

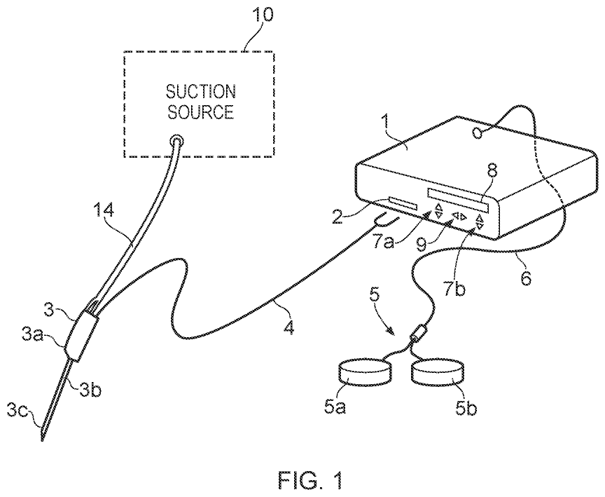

Referring to the drawings, FIG. 1 shows an electrosurgical system including an electrosurgical generator 1 having an output socket 2 providing an RF output, via a connection cord 4, for an electrosurgical instrument 3. The instrument 3 has a suction tube 14 which is connected to suction pump 10. Activation of the generator 1 may be performed from the instrument 3 via a handswitch (not shown) on the instrument 3, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

The Electrosurgical Instrument

The instrument 3 includes a proximal handle portion 3a, a shaft 3b extending in a distal direction away from the proximal handle portion, and a distal end effector assembly 3c at the distal end of the shaft 3b. A power connection cord 4 connects the instrument to the RF generator 1. The instrument may further be provided with activation buttons (not shown), to allow the surgeon operator to activate either the mechanical cutting function of the end effector, or the electrosurgical functions of the end effector, which typically comprise coagulation or ablation.

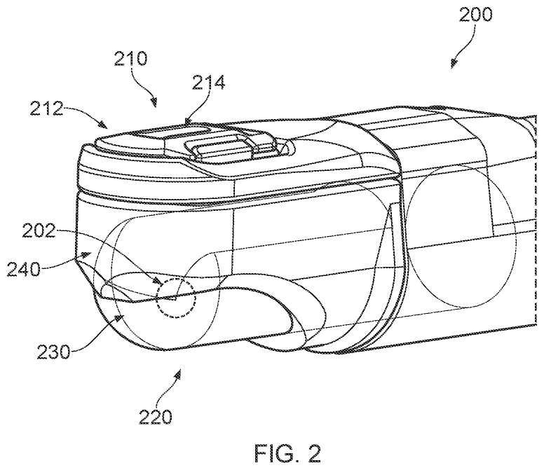

The instrument 3 may be an RF shaver instrument. An example of an RF shaver instrument is show in FIG. 2. Combining a shaver device with a RF wand is not straightforward. In prior art arthroscopic shavers, the cutting blade geometry and interface between outer blade and inner blade can be optimised for one function only—mechanical engagement and shaving of tissue. Similarly, prior art RF electrodes can optimise their suction pathways to prevent any leakage along the suction lumen and direct all available flow towards the tissue contacting active electrode area. With a dual-sided RF shaver combination device, the suction characteristics must be optimised for both modes. While shaving, the shaver window aperture and relative shearing geometry between the inner and outer blades must allow for effective tissue engagement and suction without allowing too much suction flow or pressure to be lost through the RF suction hole on the opposite side to the shave aperture. For this reason, the RF suction hole is kept as small as viable for RF use. When using the RF ablation mode, the inner blade is parked in a static ‘closed’ position (i.e., there is no overlap between the cutting windows) and all available suction flow should be directed through the RF suction aperture. The optimisation of each of these flow paths ensures that collateral tissue is not dragged into the non-active face of the device. In short, collateral tissue is not dragged towards the shaver aperture while using RF, and visa-versa. This reduces the risk of collateral tissue damage overall.

FIG. 2 shows a dual-sided RF shaver combination device 200. The device has a RF side 210 and a shaver side 220. The RF side 210 comprises an active electrode area 212. The inner blade geometry is sketched over the top of the device to show the orientation of the inner blade for illustrative purposes. In an RF shaver configuration, there are two possible suction flow paths. One through the cutting window aperture on the shaver side 220, and one through an aperture on the RF side 210. It is preferable to minimise the flow through the shave window aperture while using the RF side and minimise flow through the RF side aperture while using the shave function. This ensures that the maximum available saline suction pressure and flow rate is directed through the working suction aperture (whether that is the RF side aperture or the shave window aperture). The shaver suction window can preferably be closed during RF activation, via keeping the inner blade stationary with the teeth overlapping to close the window (i.e., such that there is no overlap between the cutting windows). For optimal suction to be directed through the RF side aperture, the shaver suction window must be closed.

For the shaver suction window to be closed, the cutting windows of the inner and outer tubular members need to be rotationally/angularly positioned such that there is no overlap between the two cutting windows. If there is the slightest overlap between the inner and outer cutting windows, an unwanted secondary suction path will form (i.e., an opening of the central suction lumen forms). FIG. 2 shows that when the inner 230 and outer 240 cutting windows slightly overlap 202, the overlap 202 results in a secondary unwanted suction flow path 202 during RF use.

In this application, a relative angular displacement between the inner and outer tubular members of 0° means the cutting windows of the inner and outer tubular members are exactly aligned. Similarly, the inner tubular member being at an angular position of 0° means the cutting windows of the inner and outer tubular members are exactly aligned. Therefore, to optimise suction during RF use, the inner and outer tubular members will ideally be rotationally positioned with a relative angular displacement of 180° (i.e., the inner tubular member will ideally be at an angular position of 180°). In other words, the cutting window of the inner tubular member will be positioned facing in a first direction and the cutting window of the outer tubular member will be positioned facing in a second direction which is exactly opposite to the first direction. This results in zero overlap between the two cutting windows, and thus no unwanted secondary suction path (i.e., the opening of the central suction lumen is not formed). However, in practice, it is difficult to precisely rotationally/angularly position the inner tubular member such that this ideal placement is achieved.

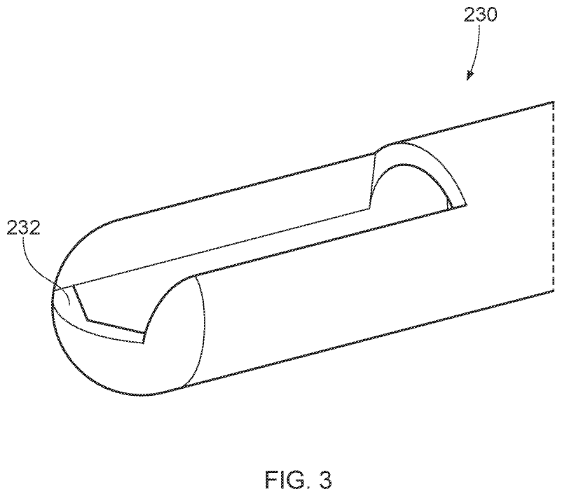

FIG. 3 shows a more detailed view of the geometry of the (unmodified) inner blade 230. Inner tubular member 230 has a square cut section or notch-shaped region 232 at its distal end which creates a rectangular-shaped opening. The opening at the distal end creates the cutting window of the inner tubular member. The edges of the cut-out of the inner tubular member form a cutting surface which, when the inner tubular member 230 rotates within the outer tubular member 240, cause tissue, bone, etc. to be cut or shaved as the cutting window of the inner tubular member/blade 230 aligns with and then becomes misaligned with the cutting window of the outer tubular member 240 as the inner tubular member is rotated within the outer tubular member. The cutting surface comprises at least one sharpened edge to form a cutting blade. The cutting blade may be serrated such that it comprises teeth.

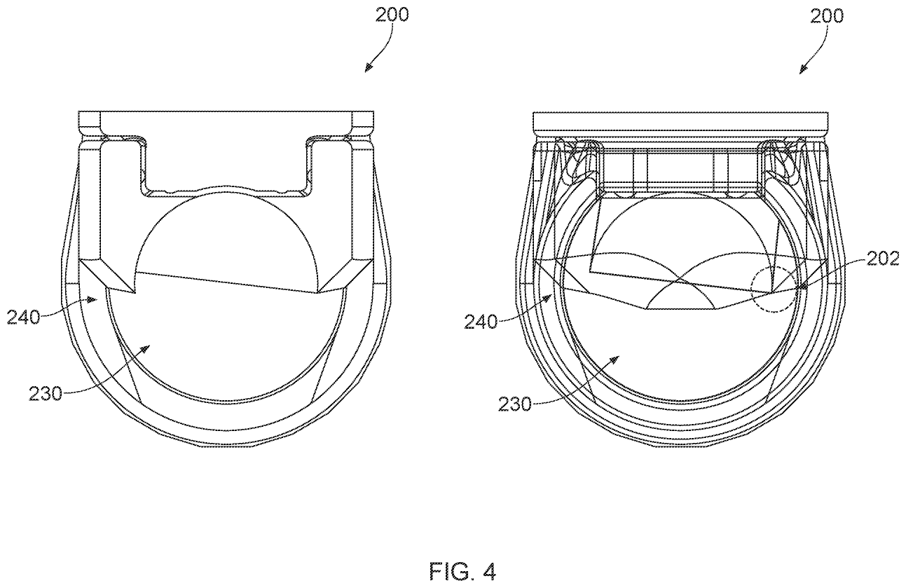

FIG. 4 is the same dual-sided RF shaver combination device 200 as FIG. 2, but the device is shown in a partial cross-sectional arrangement to show the angle at which the secondary unwanted suction flow path is formed (i.e., the angle at which the first and second cutting windows begin to overlap to form an opening of the central suction lumen). FIG. 4 shows that, for the unmodified tubular member with a cutting window comprising a notch-shaped region, at an angle of around 6° from the ideal placement (the ideal placement being a relative angular displacement of 180° between the inner and outer tubular members) the secondary unwanted suction flow path is formed. The right-hand drawing of FIG. 4 has the geometry and orientation of the inner blade 230 clearly projected on top of the cross-sectional arrangement for illustrative purposes.

FIGS. 2-4 show an arrangement with the blades being unmodified, i.e., the blades have cutting windows comprising a notch-shaped region. For these blade geometries, the inner blade 230 must be positioned or ‘parked’ with a high degree of angular accuracy to completely shut-off any leak path (also referred to as “an unwanted secondary suction path”) through the shave window aperture when using the RF modality. FIG. 2 shows an example of the small amount of overlap 202 between the inner and outer cutting windows that may cause unintended suction flow on a ‘soft tissue and bone’ style blade assembly. This is due to the corners of the notch-shaped region causing the suction path 202 to be opened when the inner tubular member is not precisely antialigned with the outer tubular member.

To ensure that the unwanted suction flow path is not formed, one approach would be to have a highly accurate blade parking system such that the inner blade can be parked within this ˜6° threshold. It would be advantageous to control the blade parking system very accurately, and also specify very tight angular tolerances in the assembly of the instrument to ensure that this ˜6° threshold is not exceeded for this blade style. The disadvantages of this approach are a higher manufacturing cost due to tight tolerances, and a more complex blade parking control system architecture as the accuracy is critical. In this application, a less costly solution is explained to achieve a similar outcome.

Overview

Embodiments of the present invention involve a modification of the geometry of a blade of a rotary shaver arrangement to prevent an unwanted secondary suction pathway to (or opening of) the central suction lumen forming at a wider (with respect to an unmodified notch-shaped blade arrangement) range of relative angular displacements between the inner and outer blades or tubular members, without increasing the assembly complexity or component cost significantly.

This can be achieved through modifying either the outer or inner blade cutting geometry, i.e., the cutting windows of the outer or inner tubular members. It is anticipated that modifying the outer blade geometry/cutting window of the outer tubular member to reduce the size of the cutting window of the outer tubular member will create a disadvantage to the surgeon, because this will mean that during shaver mode usage less tissue will be able to be cut at once by the shaver, thus preventing the surgeon from engaging with as much tissue as they would like. This may also reduce tissue resection rates. Therefore, modifying the inner blade geometry/cutting window of the inner blade is preferable, although not essential as the advantages of the invention can still be realised by modifying the outer blade cutting geometry. Were the outer blade to be modified, it would be modified in a similar manner to the modification of the inner blade as described below, i.e., the geometry of the edge of the outer blade cutting window would be modified to provide more material to block off the unwanted flow pathway by using a spline-shaped region.

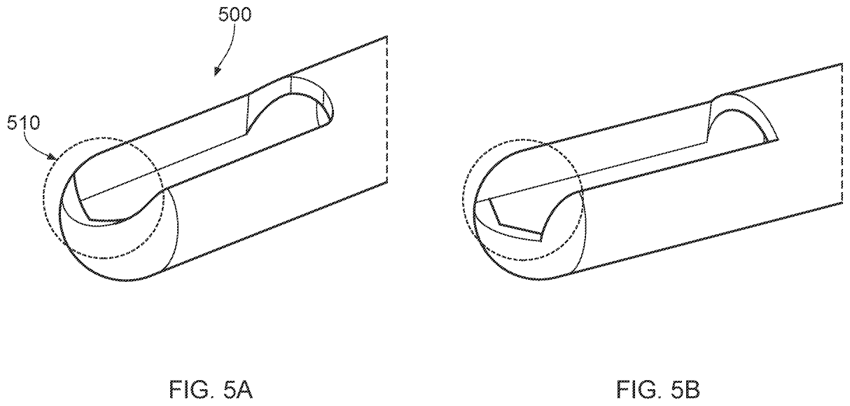

One solution proposed herein modifies the inner blade geometry over only the frontal hemispherical area which causes the secondary unwanted suction pathway, i.e., modifying the cutting window of the inner tubular member. The modification may be made only to the distal hemisphere of the inner tubular member. The modification provides more material in this area to block off the unwanted flow pathway. This is achieved with a spline-shaped region as shown in FIG. 5A to blend the geometry over the frontal hemisphere. The geometry of the blade at its distal end may be shaped such that a projection of the distal end along the longitudinal axis of the inner/outer tubular member is a continuous curve and/or non-linear. Such a projection may be part-oval. This is in contrast to the unmodified notch-shaped region as shown in FIG. 5B. The notch-shaped region has angular discontinuities instead of the smooth profile of the spline-shaped region of the modified blade.

Advantageously, the width and length of the cutting window of the modified tubular member may be unchanged from the arrangements shown in FIGS. 2-4, meaning that the ability to resect soft tissue will be unhindered compared with the unmodified blade. A prototype of this geometry was recently tested on soft tissue and bone by surgeons, and they could not reliably tell the difference between the two geometries on either soft tissue or bone when burring with the modified hemisphere. The modification of the shape of the opening/cutting window is considered to have no adverse impact on shaver performance when the inner tubular member is modified.

The angular sensitivity to opening an unwanted suction pathway during RF use is drastically reduced with the solution proposed by the present application. The sensitivity of the notch-shaped arrangement shown in FIGS. 2-4 is around ±6° and the revised spline-shaped geometry allows an angular sensitivity up to around ±33°. It may be found that increasing the angular sensitivity beyond ±33° is not advantageous because the RF suction aperture may also start to be closed off.

It is noted that one slight disadvantage to the revised geometry is that the manufacturing cost for the inner blade could be marginally more expensive, however this is deemed to be a preferred solution compared to cost associated with tightening the angular tolerances of the entire RF shaver system and all components within it, to achieve the same end.

Various aspects and details of the modified geometry of the blade will be described below by way of example with reference to FIGS. 5-8. The modified spline-shaped blade is described in comparison to the unmodified notch-shaped blade arrangement of FIGS. 2-4 for illustrative purposes.

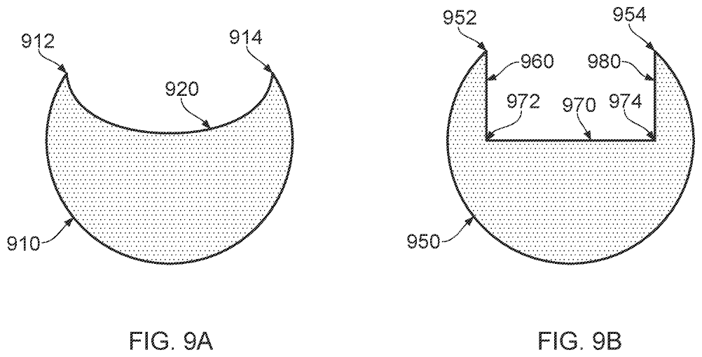

FIG. 5A shows an example of a modified inner blade 500 in accordance with embodiments of the invention. The distal end 510 of the inner blade comprises a spline-shaped feature which blends the geometry of the edge of the inner blade over the frontal (or distal) hemisphere of the inner tubular member (i.e., at the distal end 510). The distal end 510 is shaped such that a projection of the distal end along the longitudinal axis of the inner/outer tubular member has a continuous curve and/or non-linear shaped cut-out. Such a projection is illustrated in FIG. 9A where it can be seen that the projection may be shaped such that it comprises a part-oval shaped cut-out. The projection of FIG. 9A shows the shape of the cutting window is smooth. The projection of FIG. 9A has a first curve 910 and a second curve 920, the first curve 910 being from the external tubular shape of the inner tubular member and the second curve being from the smooth cut-out of the inner tubular member in order to form the cutting window. The projection of FIG. 9A has two vertices 912, 914 where the first and second curves meet.

In comparison, FIG. 5B shows an inner blade geometry which has a simple square-cut or notch-shaped distal end to create a rectangular blade opening. The projection of the distal end along the longitudinal axis of the inner/outer tubular member comprises an oblong/rectangular shaped cut-out. Such a projection is illustrated in FIG. 9B. The projection of FIG. 9B has a first curve 950 which is the external tubular shape of the inner tubular member, and then a first linear portion 960, a second linear portion 970, and a third linear portion 980, the linear portions 960, 970, 980 forming the notch-shaped or oblong/rectangular shaped cut-out. The projection of FIG. 9B has four vertices 952, 954, 972, 974. Vertices 952, 954 are where the first curve 950 meets linear portions 960, 980. Vertices 972, 974 are where linear portions 960, 980 meet linear portion 970.

The smooth curved nature of the spline-shaped cut-out of the modified inner blade geometry results in the unwanted secondary suction path being closed-off at a greater range of angular displacements (the angular displacement referring to the relative rotational angular displacement between the inner and outer tubular members) than the square nature of the notch-shaped cut-out of the unmodified inner blade.

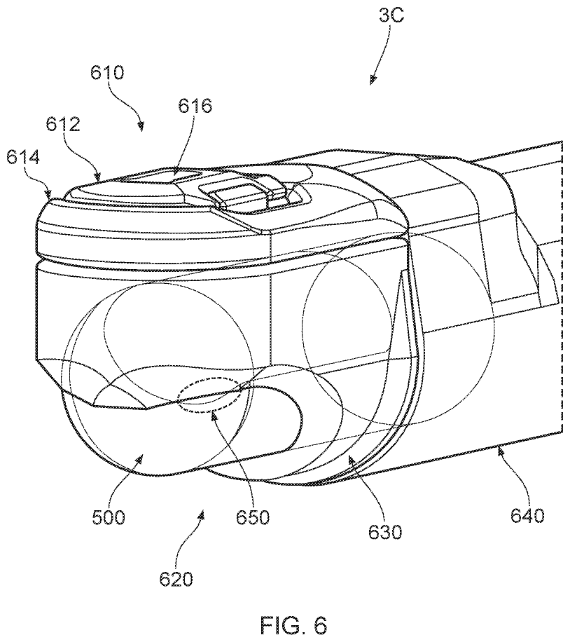

FIG. 6 shows an example of a distal end effector assembly 3c utilizing the modified inner blade 500. The example shown is a dual-sided RF rotary shaver. The distal end effector 3c has two sides to it, the RF side 610 and the shaver side 620. The shaver side 620 is on the opposite side of the end effector to the RF side 610. The shaft 3b comprises an inner tubular member which comprises the modified inner blade 500 and an outer tubular member 630. The shape and orientation of the inner blade 500 is sketched over the top of the distal end effector 3c for illustrative purposes to illustrate how the geometry of the inner blade would be orientated inside the outer tubular member 630. The outer tubular member 630 may be concentrically surrounded by an insulative tubular member 640. The inner tubular member 500 is co-axially disposed within the outer tubular member 630. The outer tubular member 630 has a larger diameter than the inner tubular member 500. The inner tubular member 500 has a proximal end and a distal end 510, with a cutting window disposed at a side of its distal end. The outer tubular member 630 also has a proximal end and a distal end, with cutting window disposed at a side of its distal end. The edges of the cutting window of the outer tubular member 630 comprise at least one sharpened edge to form a cutting blade. The at least one sharpened edge may be serrated such that it comprises teeth.

The inner tubular member/blade 500 is rotatably disposed inside of the outer tubular member 630 such that the surgical instrument 3 cuts tissue by rotating the inner tubular member 500 within the outer tubular member 630 while a vacuum is applied through the lumen of the inner tubular member 500 to draw the tissue into the cutting windows and sever the tissue by rotation of the inner tubular member 500.

The RF side 610 of the electrosurgical instrument 3 comprises an electrode assembly comprising an active electrode for tissue treatment (“active tip”) 612 received in a ceramic insulator 614. The active tip 612 may be provided with projections to concentrate the electric field at those locations. The projections also serve to create a small separation between the planar surface of the active electrode 612 and the tissue to be treated. This allows conductive fluid to circulate over the planar surface and avoids overheating of the electrode or the tissue. The active tip 612 of the instrument is provided with a suction aperture 616, which is the opening to a lumen within the inner tubular member 500.

In more detail, when the RF side 610 is to be used as a suction tool by applying a vacuum through the lumen within the inner tubular member 500, the inner tubular member 500 (which acts as a cutting blade) is stopped from rotating and the cutting windows of the inner and outer tubular members are misaligned with each other, i.e. closing the cutting windows, so that the vacuum is applied through the suction path connecting the suction aperture 616 to the suction pump 10 via the lumen to transport fluids to and from the active tip 612. It is during this use that a key advantage of the invention becomes apparent. In arrangements with unmodified blades with cutting windows having a notch-shaped region (e.g. the arrangement of FIGS. 2-4), the inner blade/tubular member has to be precisely misaligned (approximately 180°±6°) with the outer tubular member to ensure that there is no unwanted secondary suction pathway resulting from a gap between the inner and outer tubular members at the cutting window. The cutting window is only completely closed (no unwanted secondary suction pathway) when the inner tubular member is positioned at an angle of around 174° to 186° with respect to the outer tubular member. In other words, in arrangements with unmodified blades comprising a notch-shaped region, the inner tubular member must be precisely positioned in order to close the cutting window such that suction flows only to the aperture in the active tip. The angle at which the unwanted secondary suction pathway is opened is referred to as the “opening angle threshold”. Arrangements with unmodified blades have a small opening angle threshold of around ±6°.

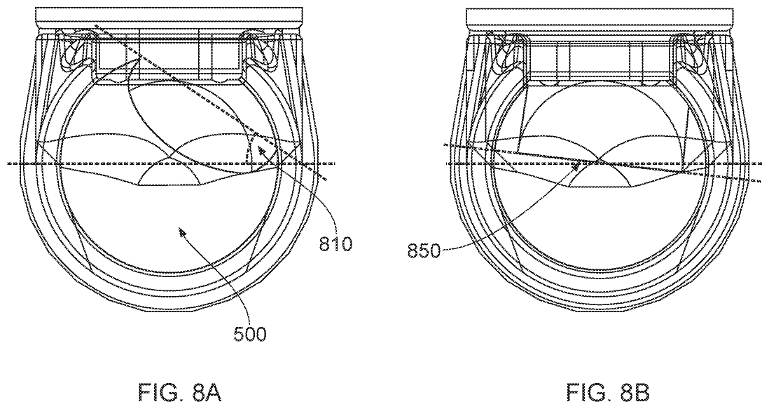

In contrast, embodiments of the present disclosure provide an inner tubular member/blade 500 with a modified geometry comprising a spline-shaped region at its distal end such that a much greater angular tolerance is provided when closing the cutting window, and therefore provide a larger opening angle threshold of up to around ±33°. For example, the cutting window can be completely closed (no unwanted secondary suction pathway) when the inner tubular member 500 is positioned at an angle of around 147° to 213° with respect to the outer tubular member 630. This is a much larger angular tolerance than that of arrangements with unmodified blades comprising a notch-shaped region. As such, there is much less need for complex blade parking control system architecture as the accuracy of the positioning of the inner blade 500 is far less critical. See FIGS. 8A and 8B which illustrate the differences in the opening angle thresholds between the modified inner blade comprising a spline-shaped region in accordance with embodiments of the invention (FIG. 8A) and the unmodified inner blade comprising a notch-shaped region (FIG. 8B). The opening angle threshold 810 of the modified blade may be up to around ±33°. The opening angle threshold 850 of the unmodified blade is typically around ±6°.

In contrast, when the shaver side 620 is in use for a cutting operation, suction may flow via through the cutting windows to the lumen, and it is preferable that it does in order to draw the tissue-to-be-cut into the cutting windows and sever the tissue by rotation of the inner tubular member 500.

The inner and outer tubular members 500 and 630 may be made from stainless steel. Stainless steel is a good option as it is easy to bond to the steel cuttings tips of inner and outer tubular members, which act as the RF return, and due to the blade properties of hardened steel.

In FIG. 6, the modified inner blade 500 is misaligned from the closed position at an angle of around 35-40° for illustration purposes. This angle is just above the opening angle threshold at which the unwanted secondary suction pathway is opened (i.e., the first and second cutting windows begin to overlap to form an opening of the central suction lumen). The unwanted secondary pathway which opens at this angle of misalignment is circled at 650.

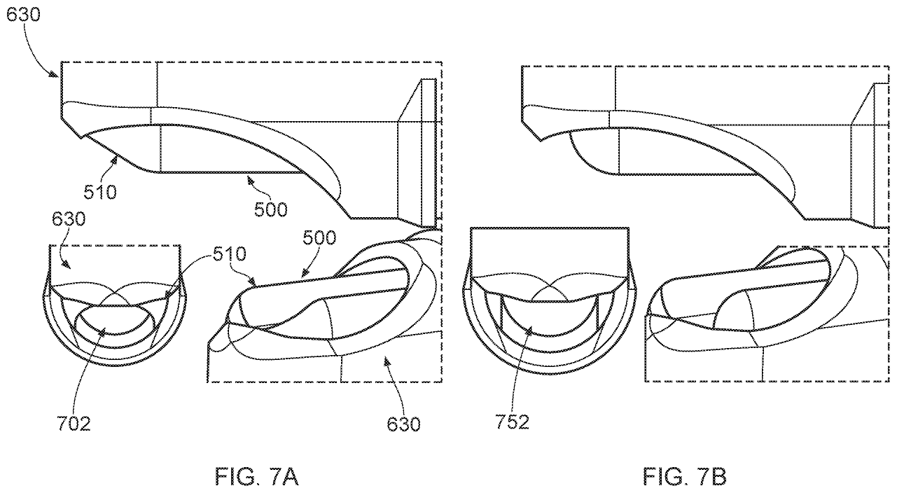

FIGS. 7A and 7B show how the inner and outer blades fit together, thereby showing the interface formed by the inner and outer blades. FIG. 7A shows an example of a modified inner blade 500 comprising the spline-shaped region in accordance with embodiments of the invention. In comparison, FIG. 7B shows an unmodified inner blade geometry comprising the notch-shaped region which has a simple square-cut distal end to create a rectangular blade opening. Both FIGS. 7A and 7B show the blades in the fully open position. The cutting windows of the inner and outer tubular members overlap to form an aperture or opening of the central suction lumen 702, 752 for the tissue to be drawn into. This aperture 702, 752 can also be referred to as an interface between the cutting windows of the inner and outer tubular members. In this position, suction flows through the aperture 702, 752 to draw tissue in such that when the inner blade 500 rotates, the tissue is severed. Although it can be seen in FIGS. 7A and 7B that the modified inner blade of the invention results in a smaller cutting window than the unmodified inner blade arrangement, the smaller cutting window has been proven to have no adverse effect on shaver performance. The shape of the interface 702 of the modified inner blade 500 instrument is round and/or oval-like in shape. In contrast, the shape of the interface 752 of the unmodified inner blade instrument is more square-like in shape. It is the extra material provided by the roundness of the interface 702 via the spline-shaped region of the cutting window which results in the larger opening angle threshold 810 of the modified blade.

FIG. 8 illustrates the difference in the opening angle thresholds between the modified inner blade in accordance with embodiments of the invention (FIG. 8A) and the unmodified inner blade arrangement (FIG. 8B). The modified inner blade of FIG. 8A has a large opening angle threshold 810 (the angle at which the unwanted secondary suction pathway is opened) compared to the unmodified inner blade of FIG. 8B. This is discussed in detail with reference to FIG. 6 above.

Reprocessing

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device may also be sterilized using any other technique known in the art, including but limited to beta or gamma radiation, ethylene oxide, or steam.

Various modifications whether by way of addition, deletion, or substitution of features may be made to above described embodiment to provide further embodiments, any and all of which are intended to be encompassed by the appended claims.

1. A rotary shaver arrangement for a surgical instrument, the rotary shaver arrangement comprising:
an outer tubular member with a first cutting window at the distal end thereof; and
an inner tubular member rotatably mounted in a central passageway of the outer tubular member, the inner tubular member providing a central suction lumen, the inner tubular member having a second cutting window at the distal end of the inner tubular member;
wherein, the first and second cutting windows are arranged such that:
(i) when the inner tubular member is rotated to a first range of angular positions, the first and second cutting windows overlap to form an opening of the central suction lumen; and
(ii) when the inner tubular member is rotated to a second range of angular positions, the first and second cutting windows do not overlap and thus do not form the opening of the central suction lumen; and
wherein, the first and/or second cutting window comprises a first shaped region, the first shaped region being a spline-shaped region to permit the second range of angular positions to be greater than if the first shaped region was notch-shaped having angular discontinuities.

2. A rotary shaver arrangement for a surgical instrument, the rotary shaver arrangement comprising:
an outer tubular member with a first cutting window at the distal end thereof;
an inner tubular member rotatably mounted in a central passageway of the outer tubular member, the inner tubular member providing a central suction lumen, the inner tubular member having a second cutting window at the distal end of the inner tubular member;
wherein, the first and second cutting windows are arranged such that:
(i) when the inner tubular member is rotated to a first range of angular positions, the first and second cutting windows overlap to form an opening of the central suction lumen;
(ii) when the inner tubular member is rotated to a second range of angular positions, the first and second cutting windows do not overlap and thus do not form the opening of the central suction lumen; and
wherein, the first and/or second cutting window comprises a spline-shaped region such that the second range of angular positions is greater than 12°.

3. A rotary shaver arrangement for a surgical instrument, the rotary shaver arrangement comprising:
an outer tubular member with a first cutting window at the distal end thereof;
an inner tubular member rotatably mounted in a central passageway of the outer tubular member, the inner tubular member providing a central suction lumen, the inner tubular member having a second cutting window at the distal end of the inner tubular member;
wherein, the first and second cutting windows are arranged such that:
(i) when the inner tubular member is rotated to a first range of angular positions, the first and second cutting windows overlap to form an opening of the central suction lumen;
(ii) when the inner tubular member is rotated to a second range of angular positions, the first and second cutting windows do not overlap and thus do not form the opening of the central suction lumen; and
wherein, the first and/or second cutting window comprises a spline-shaped region such that the inner tubular member can be rotated more than 6° from a position where the first and second cutting windows are antialigned without the opening of the central suction lumen forming.

4. The rotary shaver arrangement of claim 3, wherein the inner tubular member can be rotated more than 10, 15, 20, 25, 30 or 33 degrees from the position where the first and second cutting windows are antialigned without the opening of the central suction lumen forming.

5. The rotary shaver arrangement of claim 1 wherein the second range of angular positions is greater than 15, 20, 25, 30, 40, 50, 60 or 66 degrees.

6. The rotary shaver arrangement of claim 1, wherein the second range of angular positions comprises the inner tubular member being positioned at a relative angular position of 170° to 190° to that of the outer tubular member.

7. The rotary shaver arrangement of claim 1, wherein the spline-shaped region is such that a projection of the distal end of the inner and/or outer tubular member along the longitudinal axis of the inner and/or outer tubular member comprises a continuous curve and/or non-linear cut out.

8. The rotary shaver arrangement of claim 1, wherein the projection is part-oval.

9. The rotary shaver arrangement of claim 1, wherein the first and/or second cutting window has at least one sharpened edge to form a cutting blade.

10. The rotary shaver arrangement of claim 1, wherein the arrangement is such that, when in use, rotation of the inner tubular member within the outer tubular member causes a tissue cutting action of the cutting blade interacting with the second and/or first cutting window.

11. The rotary shaver arrangement of claim 1, wherein the second cutting window comprises the spline-shaped region.

12. The rotary shaver arrangement of claim 1, wherein the spline-shaped region is U-shaped.

13. The rotary shaver arrangement of claim 1, wherein when the first and second cutting windows align, they form a substantially oval-shaped interface.

14. An end effector for an electrosurgical instrument, the end effector comprising:
a rotary shaver arrangement according to claim 1; and
a radio frequency (RF) arrangement including an active electrode comprising a suction aperture in fluid communication with the central suction lumen.

15. The end effector of claim 14, wherein the end effector is arranged such that the RF arrangement is positioned on a first side of the end effector, and the rotary shaver arrangement is positioned such that the direction of tissue shaving of the rotary shaver arrangement is on a second side of the end effector, the second side being opposite the first side.

16. An electrosurgical instrument comprising:
an end effector according to claim 14; and
an operative shaft having RF electrical connections operably connected to the active electrode, and drive componentry operably connected to the rotary shaver arrangement to drive the rotary shaver arrangement to operate in use.

17. An electrosurgical system, comprising:
an RF electrosurgical generator;
a suction pump; and
an electrosurgical instrument according to claim 16, the arrangement being such that in use the RF electrosurgical generator supplies an RF coagulation or ablation signal via the RF electrical connections to the active electrode and the suction pump supplies suction via the central suction lumen connecting the suction aperture located within the electrode to the suction pump.
